(19) United States
(12) Patent Application Publication
(10) Pub. No.: US 2024/0374344 A1
(43) Pub. Date: Nov. 14, 2024

(54) Arthroscopic Surgery System And Method

(71) Applicant: Convergence Medical Pty Ltd., Red Hill (AU)

(72) Inventor: Christopher Arnold Jeffery, Red Hill (AU)

(73) Assignee: Convergence Medical Pty Ltd., Red Hill (AU)

(21) Appl. No.: 18/782,979

(22) Filed: Jul. 24, 2024

Related U.S. Application Data
(63) Continuation of application No. PCT/AU2024/050072, filed on Feb. 6, 2024.

(30) Foreign Application Priority Data
Feb. 6, 2023 (AU) 2023900277

(51) Int. Cl. A61B 90/50; A61B 1/00; A61B 1/015; A61B 1/317; A61B 90/98

(52) U.S. Cl. CPC A61B 90/50; A61B 1/00149; A61B 1/015; A61B 1/317; A61B 90/98

(57) ABSTRACT

An arthroscopic surgery system for assisting a surgeon in performing surgery comprising: a frame configurable to extend above or beside at least a portion of a patient to be operated; and one or more surgical arms extending from the frame, wherein each surgical arm is configured to work with a variety of different arthroscopic instruments; wherein the one or more surgical arms are arranged to assist a surgeon when performing surgery and wherein the surgical arms and attached arthroscopic instruments are controllable by the surgeon whilst performing surgery on the patient.

### TECHNICAL FIELD

The present invention relates to arthroscopic surgery. In particular, although not exclusively, the present invention relates to robotic arthroscopic surgery.

### BACKGROUND ART

Arthroscopy is a surgical procedure on a joint, such as an ankle, knee, hip, spine, shoulder, elbow or wrist, where a small camera and surgical instruments are inserted into the joint through small incisions in a patient's skin. Arthroscopy is a type of ‘keyhole’ surgery, where the surgeon is able to view the joint area and perform surgery on soft tissue such as ligaments, the bony joint surface and other tissue from the outside of the joint. In contrast to traditional “open” surgery, recovery time and tissue trauma is greatly reduced.

A problem with arthroscopy is that it is a difficult medical procedure requiring very high levels of skill. A large proportion of surgeons are not comfortable performing arthroscopy, and among the arthroscopic surgery performed, a significant portion of procedures results in observable damage caused by the procedure itself.

Certain systems exist that assist a surgeon in performing keyhole surgery for other areas of the body, including laparoscopic robots. An example of one such robot is the Da Vinci Surgical System of Intuitive Surgical, Inc. of California, United States.

In these systems, the surgeon will generally operate robotic arms from a console, the robotic arms including instruments thereon to perform the laparoscopy. Typically, the laparoscopy is performed in a large single body cavity, such as an abdomen, whereby the surgeon obtains access from a common approach. As the cavity is vast and compliant, gas is used to distend the cavity, creating a large void between the entrance and the operative site. This makes surgical visibility and access simple.

A problem with such systems is that they are not suitable for use in arthroscopy. In contrast to laparoscopy, arthroscopy involves surgery on joints, that are respectively smaller, resistant to distension and often have multiple compartments, are separated by anatomical structures, and/or are difficult to access.

As an illustrative example, laparoscopic devices are generally too large to fit into the knee. Even if laparoscopic devices were able to be made smaller, joints are mostly made of sensitive articular cartilage covering non-compliant bony surfaces, and as such, it is not possible to create a large void or cavity, analogous to that used in laparoscopy. Furthermore, even if a joint is distended, the joint space is still small and narrow, often curved and covered by cartilage that must be avoided, as bumping into, or scuffing along cartilage will wear and injure the cartilage.

As such, laparoscopic robots, such as the Da Vinci Surgical System, are not suited for use in arthroscopy.

As such, there is clearly a need for improved arthroscopic surgery systems and methods.

It will be clearly understood that, if a prior art publication is referred to herein, this reference does not constitute an admission that the publication forms part of the common general knowledge in the art in Australia or in any other country.

### SUMMARY OF INVENTION

The present invention relates to arthroscopic surgery systems and methods, which may at least partially overcome at least one of the abovementioned disadvantages or provide the consumer with a useful or commercial choice.

In one aspect, there is provided, an arthroscopic surgery system for assisting a surgeon in performing surgery comprising:
a frame configurable to extend above or beside at least a portion of a patient to be operated; and
one or more surgical arms extending from the frame, wherein each surgical arm is configured to work with a variety of different arthroscopic instruments;
wherein the one or more surgical arms are arranged to assist a surgeon when performing surgery and wherein the surgical arms and attached arthroscopic instruments are controllable by the surgeon whilst performing surgery on the patient.

In an embodiment, each of the one or more surgical arms extend from a corresponding one or more plurality of locations on the frame.

In an embodiment, the one or more surgical arms are removably coupled to the frame.

In an embodiment, the one or more surgical arms are configured to be driven along a track provided on the frame.

In an embodiment, the frame at least comprises an annular frame assembly for receiving one or more of the surgical arms.

In an embodiment, least the annular frame assembly is rotatable.

In an embodiment, the annular frame assembly further comprises intersecting frame sections passing through a geometrically central region of the annular frame assembly, the frame sections being coupled to an articulable arm during use.

In an embodiment, the intersecting frame sections are mutually perpendicular to each other.

In an embodiment, at least one of the one or more surgical arms comprises a modular end plate which is configured to engage with a variety of different arthroscopic instruments.

In an embodiment, the modular end plate includes one or more electronic, sensing and/or drive elements and/or interface elements configured to engage with corresponding elements of an arthroscopic instrument.

In an embodiment, the modular end plate comprises one or more buttons which can be used to control the surgical arm.

In an embodiment, the one or more buttons are configured to put the surgical arm into a manual mode wherein the surgical arm can be physically repositioned by a user.

In an embodiment, the arthroscopic surgery system further comprises a hand unit associated with a surgical arm and/or a modular end plate, wherein the hand unit is positioned in a user's hand for the manual movement of one or more of the surgical arms.

In an embodiment, at least one of the surgical arms has at least three degrees of freedom and more preferably at least six degrees of freedom selected from the group consisting of: (i) forward; (ii) back; (iii) up; (iv) down; (v) left; (vi) right; (vii) yaw; (viii) pitch; and (ix) roll.

In an embodiment, the arthroscopic surgery system further comprises at least one drape configured to extend over at least one of the one or more surgical arms.

In an embodiment, the one or more arthroscopic instruments include an identifier to allow the system to identify the arthroscopic instrument attached, in use, to a surgical arm.

In an embodiment, the identifier comprises an electronic tag.

In an embodiment, at least one of the arthroscopic instruments comprises a surgical camera.

In an embodiment, the surgical camera comprises a reusable base portion including an interface for providing images from the camera and a disposable distal portion releasably coupled to the reusable base portion.

In an embodiment, the arthroscopic surgery system further comprises a fluid management system including one or more pumps and valves to regulate fluid pressure and/or flow and/or visual acuity at a surgery site, wherein the one or more pumps and valves are connected to the surgery site by tubing.

In an embodiment, the arthroscopic surgery system further comprises one or more consumables wherein the one or more consumables comprise a readable identifier and wherein the system further comprises a reader for reading the readable identifier and determining whether the one or more consumables are genuine consumables.

In another aspect, there is provided an arthroscopic surgery apparatus for assisting a surgeon in performing surgery comprising:
a frame configurable to extend above or beside at least a portion of a patient to be operated; and
one or more surgical arms extending from the frame, wherein each surgical arm is configured to work with a variety of different arthroscopic instruments;
wherein the one or more surgical arms are arranged to assist a surgeon when performing surgery and wherein the surgical arms are controllable by the surgeon whilst performing surgery on the patient.

In another aspect, there is provided a method of arthroscopic surgery, the method comprising the steps of:
providing an arthroscopic surgery apparatus comprising a frame and one or more surgical arms extending from the frame;
attaching an arthroscopic instrument to one or more of the surgical arms;
positioning the frame and/or attached arthroscopic instrument to extend above or beside at least a portion of a patient to be operated; and
utilising the one or more surgical arms to assist a surgeon when performing surgery;
wherein the method further comprises the surgeon controlling movement of the surgical arms and/or attached arthroscopic instruments whilst performing surgery on the patient by accessing a controller located at or adjacent the surgical arms.

In another aspect, there is provided an arthroscopic surgery system for assisting a surgeon in performing surgery comprising:
a frame configurable to extend above or beside at least a portion of a patient to be operated;
one or more surgical arms extending from the frame, wherein each surgical arm comprises a modular end plate configured to work with a variety of different arthroscopic instruments;
one or more arthroscopic instruments coupled, in use, to the one or more surgical arms via a modular end plate;
wherein each modular end plate is configured to provide mechanical control and/or electrical control of an arthroscopic instrument for surgery

In yet another aspect, there is provided a method of performing arthroscopic surgery comprising:
providing an arthroscopic surgery system for assisting a surgeon in performing surgery, wherein the arthroscopic surgery system comprises a frame configurable to extend above or beside at least a portion of a patient to be operated and one or more surgical arms extending from the frame, wherein each surgical arm comprises a modular end plate configured to work with a variety of different arthroscopic instruments;
attaching an arthroscopic instrument to each surgical arm via a modular end plate, wherein the arthroscopic instrument comprises an identifier;
identifying the arthroscopic instrument utilising the identifier; and
mechanically controlling and/or electrically controlling an arthroscopic instrument via the modular end plate.

In another aspect, the present disclosure resides broadly in an arthroscopic surgery system including:
a frame, configurable to extend above or beside at least a portion of a patient to be operated; and
one or more surgical arms, extending from the frame, the one or more surgical arms each including an arthroscopic instrument;
wherein the one or more surgical arms and associated arthroscopic instruments are remotely controllable from a user interface to perform arthroscopic surgery.

Advantageously, the system simplifies arthroscopic surgery through the use of remotely controllable instruments, and thereby reduces the likelihood of damage or injury being caused from the surgery itself. Furthermore, as the system is simpler to use than traditional arthroscopic instruments, the barrier for entry into arthroscopy is reduced.

The use of the frame enables the surgical arms, and the associated arthroscopic instruments, to operate with reference to a single origin (defined with reference to the frame), and thereby enables simple cartesian mapping of locations.

Preferably, the frame is rigid. Preferably, the frame extends laterally in two dimensions.

Preferably, the frame is configurable to extend laterally above at least a portion the patient. Preferably, the at least one surgical arm is reconfigurable to extend from different lateral positions on the frame. Preferably, the one or more surgical arms comprise a plurality of surgical arms that extend from different lateral positions on the frame.

As the frame extends laterally, and the surgical arms extend downwardly from different lateral positions, multiple portal sites, e.g. on different sides of the joint, may be used, while retaining a common coordinate system between the surgical arms (unlike independent arms that are not connected by a frame).

In some embodiments, the frame is annular. The surgical arms may extend from a periphery of the frame.

In some embodiments, the frame is adjustable. This may be achieved by adding portions to the frame or, by removing portions of the frame.

Preferably, the one or more surgical arms comprises a plurality of surgical arms.

Preferably, the surgical arms are re-configurable to extend from the frame at a plurality of different positions. In some embodiments, the surgical arms are configurable to extend from the frame at one or a plurality of predefined positions. The surgical arms may be removably coupled to the frame.

Alternatively, the surgical arms may be moved relative to the frame, e.g. using actuators, a drive assembly or the like. The arms may be moved based upon user input. An end of the surgical arm may be secured to the frame using a track. Preferably, the first end of the arm is configured to be driven along the track. In some embodiments, a plurality of tracks may be employed. In such instance, it is envisioned that the ends of the surgical arms may be stacked vertically with different arms residing in each track.

The frame may include one or more roller assemblies for movement of the surgical arms along the track. Preferably, the one or more roller assemblies are received across and along the track. Each guide track may include at least one channel for guiding passage of the roller assemblies. As indicated, it is envisioned that a plurality of guide tracks are employed. In such instance, one roller assembly of the one or more roller assemblies may be configured to engage with one of the plurality of guide tracks.

Preferably, the first end of the at least one arm includes an attachment portion for attachment to the frame. Preferably, the attachment portion is attached to the frame by screws, pins or similar fasteners. In some embodiments, the attachment portion is configured to clamp to or otherwise engage with the frame.

The arms may be of different lengths to achieve varied functions. For instance, different length arms may offer engagement with different parts of a patient, or may be used to reach a patient in varied operative positions.

Preferably, the surgical arms are coupled to the frame both electrically and mechanically.

Preferably, the surgical arms are then able to articulate into a variety of different positions based upon user input.

Preferably, the surgical arms are configured to bend around at least one pivot point. Preferably, the surgical arms are configured to bend around two or more pivot points.

Preferably, at least part of the surgical arm is configured to rotate.

Preferably, at least one of the surgical arms has at least six degrees of freedom. The six degrees of freedom may include one or more of forward/back, up/down, left/right, yaw, pitch, and roll.

In some embodiments, the surgical arms have 8 degrees of freedom.

Preferably, surgical arms can be added or removed from the frame in a modular manner.

Preferably, the surgical arms have modular end plates, configured to work with a variety of different instruments.

Preferably, the modular end plates have one or more buttons that can be used to control the surgical arms. In some embodiments, the buttons may be used to put the arms into a manual mode so they can be physically repositioned by users. Similarly, the buttons may be used to save points into the runtime program.

The modular end plates may include drive elements, which are configured to engage with corresponding drive elements of an instrument, to mechanically drive one or more aspects of the instrument.

The modular end plates may include electrical elements, configured to electrically couple the instrument to the system. The electrical elements may be configured to power the instrument, and/or receive or transmit data to or from the system.

The modular end plates may each include powered drive elements and electrical contacts at the same predefined positions, to enable the use of a wide range of instruments in a modular manner. The arthroscopic instruments may then use any one or more of these powered drive elements and/or electrical contacts, based upon need.

Preferably, a hand unit is associated with the surgical arms and/or a modular end plate. The hand unit positioned in a user's hand for the manual movement of one of more of the surgical arms. Preferably, the hand unit is operatively associated with the one or more surgical arms such that movement of the hand unit associated with one of the surgical arms and/or modular end plate causes movement of the remaining one or more surgical arms.

The hand unit is configured to fit in a user's hand for manual operation by the user. The hand unit may fit in a user's hand in a typical manner such as for example, in a user's palm or fingers.

In use, it is envisioned that a user may position the surgical arms using the hand unit. Advantageously, this may enable the user to manually orient the surgical arms at an appropriate site of the patient which may in turn assist with surgeon liability concerns.

The hand unit may further include a locking mechanism. Preferably, the locking mechanism is configured to lock the surgical arms for operation within a region relative to the patient.

Preferably, the hand unit is configured to generate haptic signals. The haptic signals may be generated according to data from the system, and may correspond to the movement and locking of the hand unit, or any other suitable information.

In alternative embodiments, one or more of the arthroscopic instruments are permanently or semi-permanently attached to an associated surgical arm. The arthroscopic instruments may be attached to the arms by fasteners (e.g. nuts and bolts), or may form part of the surgical arms.

The system may include drapes, extending over each of the surgical arms. The drapes may be sterile. The drapes may include rigid upper and lower mounts, and a flexible sheath extending therebetween. The rigid upper and lower mounts may include apertures, through which connectors of the arm and/or instruments extend.

The rigid upper mount may be adapted to be received intermediate the frame and the surgical arm and the rigid lower mount may be adapted to be received intermediate the arm and the arthroscopic instrument.

Preferably, the flexible sheath provides a continuous tube between the apertures of the rigid upper and lower mounts. In one embodiment flexible sheath includes tubing. The tubing may be internal the flexible sheath tube, or positioned outside the flexible sheath tube. Preferably, the tubing connects with one or more pumps and valves to regulate fluid pressure and flow. The one or more pumps may comprise a series of pumps. The valves may comprise compression valves.

The drapes may include tubing which is connectable to a fluid inflow/outflow system, suction system, data and power and/or an RF energy system.

The skilled addressee will appreciate that the drapes are single use. Preferably, the drapes are provided in a packaging for use with the system. The packaging may be any suitable type to maintain sterility.

Preferably, the drapes are provided in a tear open sterile packaging such that when the packaging is torn open, the drapes therein remain sterile.

In embodiments where the drapes include rigid upper and lower mounts, the packaging may further include one or more packaging chambers. The one or more packaging chambers may be configured to cover the rigid upper and lower mount, and the flexible sheath separately such that the rigid upper and lower mounts may be removed from the packaging separately so as to reduce risk of contamination.

Preferably, the instruments include a modular coupling portion. The modular coupling portion may include a plurality of drive elements, which are configured to engage with corresponding drive elements of the modular end plates of the arms. The system may then control the arthroscopic instrument through rotation of the drive elements on the modular end plates.

The modular coupling portion may include a plurality of electrical coupling elements, which are configured to engage with corresponding electrical elements of the modular end plates of the arms. The electrical elements may be used to power or communicate with the instrument (to and/or from the instrument).

The instruments may include internal cabling, coupled to gears of the drive elements and internal pulleys, to cause the instrument to bend and actuate.

The instruments may include identifiers to allow the system to identify the attachments. The identifiers may comprise RFID tags.

The instruments may include an arthroscopic camera.

The arthroscopic camera may be configured to capture images. The arthroscopic camera may be configured to send electrical signals back to the system in the form of live/streaming video imagery. The arthroscopic camera may include a light source which is powered from the robot arm.

Preferably, the arthroscopic camera is configured to be inserted into the operative site. In this regard, it is envisioned that the arthroscopic camera captures imagery of the operative site from within the operative site.

Preferably, the arthroscopic instruments are less than about 5 mm in diameter at a portion. Preferably, the arthroscopic instruments are less than about 5 mm in diameter at the portion of the arthroscopic instrument to be inserted into the operative site. The arthroscopic instrument may be between about 1.9 mm-4.0 mm in diameter. Similarly, the instruments may be between about 90 mm-170 mm length.

The arthroscopic instruments may be in electronic communication with a controller of the system by one or more electrical cables, cords or the like. More preferably, the surgical effector and the controller may be in wireless communication with the controller (for instance, by Bluetooth, near field communication (NFC), Wi-Fi or the like).

The system may include a Radio Frequency (RF) energy generator, configured to generate RF energy for use by one or more of the instruments.

Preferably, the system is configured to generate a depth map from images captured by the system. The depth map is preferably used to create 3D video from the images captured by the mono-optic camera.

Preferably, depth data associated with the depth map is overlaid over image data.

Preferably, contour lines are overlaid over the image data.

Preferably, the image is processed. The image may be processed to adjust or improve contrast, focus, white balance and/or resolution.

The image may also be flipped, rotated and/or scaled with/without user surgeon input. For example, the image may be automatically flipped, rotated and/or scaled. Alternatively, the surgeon may rotate the image such that the surgeon is working in a comfortable (e.g. upright) position, regardless of the orientation of the surgical instruments.

In some embodiments, the image data is classified. One or more labels may be shown in association with the classification.

Preferably, the system is configured to automatically identify “not normal anatomy”, and label same, e.g. using image overlays or the like.

In some embodiments, training data from past cases is used to create identification classifiers (e.g. through Haar classifiers or similar). These identification classifiers may then be used to automatically classify anatomy of the joint.

Preferably, the system includes a fluid management system. The fluid management system may be configured to regulate the flow of fluid, pressure, or temperature to the joint either through the instruments or directly. The fluid may comprise saline.

The fluid may be configured to prevent heat dissipation into surrounding tissue, e.g. in the case of cauterisation or other heat generating instruments.

One or more sensors, such as temperature sensors, may be provided on the instrument. Data from the sensors may be provided to the system.

The fluid management system may adjust flow rates according to data from the sensors.

The fluid management system may include a pump, pressure sensors and control valves (e.g. pinch valves) to regulate pressure and flow to the instruments. The fluid management system may include one or more lines. In the case of multiple lines, each line may be regulated independently.

The fluid management system may be configured to automatically maintain temperatures and pressures within certain thresholds.

The system may include a suction system, that is used for fluid outflow, and may be coupled to an external suction system, which manages the suction and handling of fluids.

The fluid management system may be configured to distend a joint on which arthroscopic surgery is performed.

Preferably, distention is achieved by regulating a pressure and/or flow of fluid in the fluid management system. The pressure and or flow may be regulated at least in part according to sensor data. The sensor data may include inflow pressure and/or outflow pressure. The system may be configured to use a model of the joint and/or system to regulate pressure and/or flow.

Preferably, the system is at least partially portable. The system may include a wheeled base. The wheeled base may be lockable into position.

Preferably, the frame is supported in an elevated position by an articulable arm that extends upwardly from the wheeled base.

In some embodiments, the surgical system includes a storage site for storing auxiliary items, wiring and other infrastructure and/or consumables.

Preferably, the storage site is located at the base of the robot. The storage site may further include shelving, draws, or like compartments to assist with organisation. The storage site may be lockable.

Preferably, the storage site stores a suction pump, regulator, fluid trap, and the like. In this regard, the storage site may house a portion of the fluid management system.

In some embodiments, the base includes a locking mechanism for locking the base in position. The locking mechanism may for example be a foot pedal. It is envisioned that the base may be wheeled in to an appropriate position fist, and locked in position using the locking mechanism. Advantageously, this may prevent accidental movement of the base and thereby the arthroscopic instruments during operation such as via bumping by surgeons or other medical professionals, or through environmental factors such as earthquakes.

As indicated, consumables may be positioned in the storage site. Preferably, the consumables include readable identifiers such as for example, a serial number, barcode, or QR code. The readable identifiers may be read by the system. Preferably, the readable identifiers may be read by a reader. Preferably, the reader is associated with the modular end plates on the arms of the robot.

It is envisioned that the reader may detect whether the consumables are genuine consumables associated with the system. The system may restrict use where non-genuine consumables are detected.

Preferably, the use of consumables is recorded. This use may be recorded by the operation of the system, that is, use may be recorded when the system is in operation. Alternatively, or in conjunction, the use may be recorded when the consumable is read by the reader associated with the modular end plates.

Advantageously, recording the use of consumables may enable the system to identify consumable stock levels. The system may be configured to access the Internet to automatically place an order for consumables.

An order interface may display an order for consumables and require a user and/or hospital procurement department to confirm the order for consumables before it is placed. Preferably, however, the order interface is separate the user interface for remotely controlling the arthroscopic surgery robot.

In alternate embodiments, the system may be configured to generate a report outlining the consumable stock levels for consideration by a user and/or a hospital procurement department.

Preferably, the system is configured to generate a map, showing relative positioning of the instruments. Preferably, the map includes a relative position of the patient.

Preferably, the system includes a headset. Preferably, the system includes one or more controllers. The surgeon may interact with the one or more controllers to control the instruments, and thus perform surgery.

Preferably, image data from an arthroscopic camera is displayed on the headset.

Preferably, the controllers are configured to control operation and movement of the camera and instruments.

In some embodiments, the system may be configured to generate haptic signals through the controllers.

The haptic signals may be generated according to data from the system, and may correspond to warnings, confirmations, or any other suitable information.

Preferably, the system includes a console, comprising a display and one or more user input devices, to enable a user to interact with the system. The user input devices may be in the form of joystick controllers and/or a keyboard.

The system may include a central controller to control various aspects of the system. The central controller may comprise one or more processors, memory coupled to the processors, the memory including various instruction code to perform the functions of the system.

In another form, the invention resides broadly in a surgery system including:
one or more surgical arms, each including a surgical instrument;
an intraoperative fluid management system associated with at least one of the one or more surgical arms and/or surgical instruments;
wherein the one or more surgical arms and associated surgical instruments are remotely controllable to perform arthroscopic surgery.

The fluid management system may include one or more pumps and valves to regulate fluid pressure and/or flow. The one or more pumps may comprise a series of pumps. The valves may comprise compression valves.

The fluid management system may be configured to regulate the flow of fluid, pressure or temperature of fluid to the body either through the instruments or directly. The fluid may comprise saline.

The fluid may be configured to prevent heat dissipation into surrounding tissue, e.g. in the case of cauterisation or other heat generating instruments.

One or more sensors, such as temperature sensors, may be provided on the instruments. Data from the sensors may be provided to the fluid management system.

The fluid management system may adjust flow rates according to data from the sensors.

The fluid management system may regulate pressure and flow to the instruments. The fluid management system may include one or more lines. In the case of multiple lines, each line may be regulated independently.

The fluid management system may be configured to automatically ensure that temperatures and pressures remain within certain thresholds.

In yet another form, the invention resides broadly in a surgical drape for mounting to an object during surgery, the surgical drape including:
a sheath configured to at least partially cover an object; and
at least one tube, for transporting fluid to or from a patient during surgery.

Preferably, the drape is configured to mount to a remotely controllable surgical arm of a surgical system. The drape may extend over the surgical arm.

Preferably, the sheath is flexible.

The drape may further include rigid upper and lower mounts, wherein the sheath extends therebetween. The rigid upper and lower mounts may include apertures, through which connectors of the arm and/or instruments extend.

Preferably, the flexible sheath provides a continuous tube between the apertures of the rigid upper and lower mounts.

The rigid upper mount may be adapted to be received intermediate a frame and the surgical arm and the rigid lower mount may be adapted to be received intermediate the arm and a surgical instrument.

Preferably, a tube of the at least one tube is configured to transport intraoperative fluid. The intraoperative fluid may comprise saline.

In yet another form, the invention resides broadly in an arthroscopic instrument including:
a shaft including a distal end, the distal end configured to enter a joint during surgery; and
an articulable joint, located adjacent to a tip of the shaft at the distal end, the articulable joint configured to enable the tip of the shaft to change pose.

Preferably, the articulable joint includes a pivot joint. Preferably, the articulable joint includes a rotation joint. The articulable joint may include a pivot and a rotation joint.

Preferably, the rotation joint is a ball and socket joint. The ball may be at least partially received in an end of a surgical arm. Preferably, the ball is rotatably secured in position with an end cap extending partially over the ball at an opposed end to the surgical arm. Supports may extend between the end cap and the surgical arm to retain the ball in position. Preferably, the supports are substantially elongate.

Preferably, the supports are resilient such that they may move to enable movement of the ball contained therein. In this regard, the supports may be made of an elastomer polymer.

In some embodiments, the supports comprise two or more support members. In some such embodiments, a first support member may be associated with a second support member by their respective ends and so on.

The arthroscopic instrument may include one or more cables, configured to cause movement of the articulable joint. The cables may be coupled to one or more pulleys.

Preferably, the cables extend along a length of the shaft. Preferably, the cables extend internally in the shaft.

The arthroscopic instrument may include one or more drive elements, coupled to the one or more cables. Preferably, rotation of the one or more drive elements may cause translation of the one or more cables in the shaft. Preferably, the drive elements are located at or adjacent to a proximal end of the shaft.

Preferably, the instrument includes a modular coupling portion. The modular coupling portion may include a plurality of drive elements. The modular coupling portion may include a plurality of electrical coupling elements.

The instrument may include a marker to allow identification of the instrument. The marker may comprise an RFID marker.

In yet another form, the invention resides broadly in a surgical camera including:
a reusable base, including an interface for providing images from the camera; and
a disposable distal portion, releasably coupled to and extending outwardly from the reusable base.

Preferably, the disposable distal portion is at least partly configured to enter the body of a patient during surgery. In one embodiment, the camera is an arthroscopic camera. At least part of the distal portion is configured to enter a joint during surgery.

Preferably, the disposable distal portion includes an image sensor. Preferably, the disposable distal portion includes one or more articulable joints.

Preferably, the reusable base and the disposable distal portion are electrically coupled. Preferably, the reusable base and the disposable distal portion are mechanically coupled. The reusable base and the disposable distal portion may be mechanically coupled by one or more drive members, such as cogs or gears.

The disposable distal portion may be single use. The disposable distal portion may be multi-use.

In other embodiments, the camera may include a disposable cover on at least a distal portion thereof.

In another form, the invention resides broadly in a surgery system including:
a camera, configured to capture images during surgery; and
a processor, configured to generate depth data according to the captured images.

The camera may comprise a mono-optic camera. The camera may be configured to enter the body of a patient during use, that is, during operation.

Preferably, the system is further configured to overlay depth data, or a derivative thereof, on one or more of the captured images for display. The system may be configured to overlay depth cues, such as contour lines, on the images.

The system may be configured to overlay operative information, and/or equipment information. As an illustrative example, the system may be configured to overlay pre-operative planning information, runtime data, such as distances or angles, system performance information, such as instrument loads, usability information, or any other suitable and relevant information.

The system may include a display on which the captured images with overlaid depth data, or derivative thereof, are displayed. The display may be configured to display the overlaid images in or near real time.

The system may comprise a surgical instrument, on which the camera is provided. The camera may comprise an arthroscopic camera.

The camera may be configured to capture video as the camera moves relative to a patient. The depth data may be generated, at least in part, according to differences between images captured by the camera at different points in time.

The system may include a dedicated Camera Control Unit (CCU), for managing the camera signal and to control the camera sensor. Various image processing functions may be performed using the CCU.

The camera may include an LED light source associated therewith, to illuminate an area being captured. The LED light source may be adjustable, and may be associated with a tip of the camera.

The light source may be positioned within the camera. Preferably, the light source is positioned adjacent the camera. Most preferably, the light source is positioned adjacent the camera and may be associated with the surgical arm to which the camera is mounted.

In alternate embodiments, the light source is separately mounted from the camera. Preferably, the light source is mounted on a moveable member associated with the surgical arm to which the camera is mounted. Advantageously, this may enable the camera to be separately oriented to illuminate the view of the camera without casting shadows, or obstructing the operative site.

In yet another form, the invention resides broadly in an arthroscopic surgery system including:
an arthroscopic camera, configured to capture images during surgery; and
a headset, configured to display the captured images.

The headset may be configured to display a three-dimensional image. The three-dimensional image may comprise a stereoscopic image. The stereoscopic image may be generated from mono images from the arthroscopic camera.

The arthroscopic surgery system may include controllers, through which a surgeon may interact.

In yet another form, the invention resides broadly in an arthroscopic surgery system including:
one or more surgical arms, each including an associated arthroscopic instrument; and
a handheld controller including more than three degrees of freedom, wherein the one or more surgical arms and associated arthroscopic instruments are remotely controllable by the handheld controller to perform arthroscopic surgery.

The handheld controller may include one or more joysticks.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 illustrates an arthroscopic surgery system, according to an embodiment of the present invention.

FIG. 2 illustrates a side view of an arthroscopic instrument of the system of FIG. 1, in the form of an arthroscopic grasper, according to an embodiment of the present invention.

FIG. 3a illustrates a side view of an arthroscopic instrument of the system of FIG. 1, in the form of an arthroscopic camera, according to an embodiment of the present invention.

FIG. 3b illustrates a side view of the arthroscopic instrument of FIG. 3a, in a separated configuration, according to an embodiment of the present invention.

FIG. 4 illustrates a side view of an articulable joint of the system of FIG. 1, according to an embodiment of the present invention.

FIG. 5a illustrates a perspective view of a sterile drape of the system of FIG. 1, according to an embodiment of the present invention.

FIG. 5b illustrates a simplified side view of the sterile drape of FIG. 5a, according to an embodiment of the present invention.

FIG. 6 illustrates a simplified view of an end plate of the system of FIG. 1, according to an embodiment of the present invention.

FIG. 7a illustrates a perspective view of an end plate for an arthroscopic instrument of the system of FIG. 1, according to an embodiment of the present invention.

FIG. 7b illustrates a perspective view of an attachment portion for an arthroscopic instrument of the system of FIG. 1, according to an embodiment of the present invention.

FIG. 7c illustrates an exploded view of the end plate and attachment portion of FIGS. 7a and 7b, according to an embodiment of the present invention.

FIG. 8a illustrates a screenshot of a camera image from the system of FIG. 1, without any image overlay.

FIG. 8b illustrates a screenshot of a camera image from the system of FIG. 1, with contour lines overlaid onto the image.

FIG. 9 illustrates a screenshot comprising four different image screens of the system of FIG. 1 (i.e. images viewed by the surgeon during surgery), according to embodiments of the present invention.

FIG. 10 illustrates a perspective view of a portion of an arthroscopic surgery system, according to an alternative embodiment of the present invention.

FIG. 11 illustrates a screenshot of a surgical screen of an arthroscopic surgery system, according to an embodiment of the present invention.

FIG. 12 illustrates an upper perspective view of an attachment portion of the system of FIG. 1, according to an embodiment of the present invention.

FIG. 13 illustrates a rear view of an arthroscopic instrument, of the system of FIG. 1, in the form of a hand grip, for connection with the attachment portion of FIG. 12, according to an embodiment of the present invention.

FIG. 14 illustrates a side view of an arm of an arthroscopic instrument of the system of FIG. 1, according to an embodiment of the present invention.

FIG. 15 illustrates side view of a frame of an arthroscopic surgery system of FIG. 1, according to an embodiment of the present invention.

FIG. 16 illustrates an enlarged side view of a portion of the frame of FIG. 15, according to an embodiment of the present invention.

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

### DESCRIPTION OF EMBODIMENTS

Embodiments of arthroscopic surgery methods and systems are described below, which simplify arthroscopic surgery, and thereby reduce likelihood of damage or injury being caused from the surgery itself. Furthermore, as the methods and systems are simpler to use than traditional arthroscopic instruments, the barrier for entry into arthroscopy is reduced.

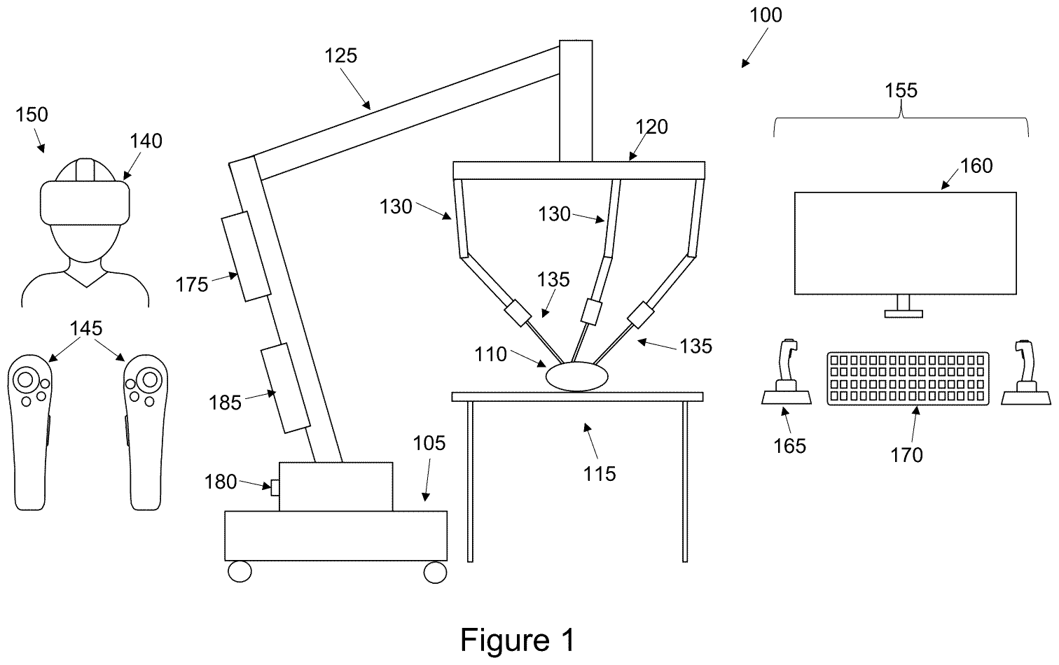

FIG. 1 illustrates an arthroscopic surgery system 100, according to an embodiment of the present invention.

The arthroscopic surgery system 100 is portable, and includes a wheeled base 105, which may be moved to a desired location in an operating theatre, and locked into place. In use, the wheeled base 105 will be moved into position adjacent to a patient 110 on an operating table 115 in preparation for surgery, and locked at that position prior to, and during the duration of, surgery performed using the system 100.

The system 100 includes a rigid frame 120, supported in an elevated position by an articulable arm 125 that extends upwardly from the base 105. The frame 120 includes a plurality of surgical arms 130, each including a plurality of remotely operable arthroscopic instruments 135, as outlined in further detail below.

The system 100 is for arthroscopic surgery, i.e. surgery on a joint, such as an ankle, knee, hip, spine, shoulder, elbow or wrist. In use, and after the wheeled base 105 is locked into position, the frame 120 is positioned above the joint to be operated on of the patient 110. The frame 120 extends laterally above the patient, which enables the surgical arms 130 and arthroscopic instruments 135 to operate from different sides of the joint, while having a common reference point through the rigid frame 120.

The system 100 includes a headset 140 and controllers 145, operable by a surgeon 150, to remotely control the surgical arms 130 and arthroscopic instruments 135. In particular, the arthroscopic instruments 135 include an arthroscopic camera and instruments, such as cutters and graspers.

The controllers 145 have more than three degrees of freedom (e.g. up/down, left/right and forward/backward). In preferred embodiment, the controllers 145 have at least 6 degrees of freedom. The degrees of freedom may be provided through movement of the controller itself, and/or interaction with components thereof.

Image data from the arthroscopic camera is processed and displayed on the headset 140 to the surgeon 150, and the controllers 145 control operation and movement of the camera and instruments 135. As such, the headset 140 and controllers 145 may function like a virtual reality (VR) system.

The headset 140 may be configured to display a three-dimensional image, such as a stereoscopic image, which may be generated according to depth data estimated from the images.

The system 100 may be configured to generate haptic signals through the controllers 145, to assist the surgeon 150. The haptic signals may be generated according to data from the system 100, and may correspond to warnings, confirmations, or any other suitable information.

Similarly, the system 100 may be configured to generate and display on the headset 140 a positional map, showing relative positioning of the instruments 135 and the patient 110. The system 100 may allow the surgeon 150 to zoom in and out, and rotate the model, enabling the surgeon 150 to operate from various angles in a common (e.g. upright) position.

The system 100 further includes a console 155, comprising a display 160 and user input devices in the form of joystick controllers 165 and a keyboard 170. The console 155 can be used to perform the procedure or in a reduced capacity used to adjust parameters of the system 100, prior to surgery, to enter patient details, and/or to assist the surgeon 150 during surgery. The headset 140 and the console 155 are preferably set up for concurrent operation.

The skilled addressee will, however, readily appreciate that any number of and type of suitable user interfaces may be used. As an illustrative example, the console 155 may be used by the surgeon 150 rather than the headset 140 and controllers 145.

As outlined above, arthroscopic surgery generally requires the use of multiple portal sites. The surgical arms 130, and thus the arthroscopic instruments 135, are positionable on the frame 120 such that they may be positioned on various sides of the joint being operated on.

The frame 120 is generally wide and extends laterally in two dimensions. In one embodiment, outlined in further detail below, the frame 120 is annular, and the surgical arms 130 are positionable around the frame.

In some embodiments, the surgical arms 130 are positionable in a plurality of predefined positions around the frame 120. In other embodiments, the surgical arms 130 may be moved relative to the frame, e.g. using actuators, a drive assembly or the like, e.g. based upon user input.

The surgical arms 130 themselves are then able to articulate into a variety of different positions based upon user input. In some embodiments, the arms 130 include robotic wrists with at least one horizontal and two vertical pivots, thereby providing 8 degrees of freedom. The arms 130 may be configured to bend at an end thereof, to enable the “pose” of the end of the instrument to be changed.

Such configuration enables the arthroscopic instruments 135 to move in all Cartesian directions and rotate around all planes, to access the joint from desired angles.

While the system 100 of FIG. 1 illustrates three surgical arms 130, surgical arms 130 may be added and removed according to need. The skilled addressee will thus readily appreciate that any suitable number of arms may be used.

The arthroscopic instruments 135 are coupled to the arms 130 using modular end plates. The modular end plates include a plurality of motors (e.g. 3 motors) to drive aspects of the arthroscopic instruments 135, and a plurality of electrical contacts, for powering, controlling and/or communicating with the arthroscopic instruments 135. Such configuration enables a wide range of arthroscopic instruments 135 to be used with the system 100 in a modular manner, and without needing to modify the arms 130.

While motors (mechanical control) and electrical contacts (electrical control) are described, the skilled addressee will readily appreciate that some components may be entirely mechanical and/or electrical.

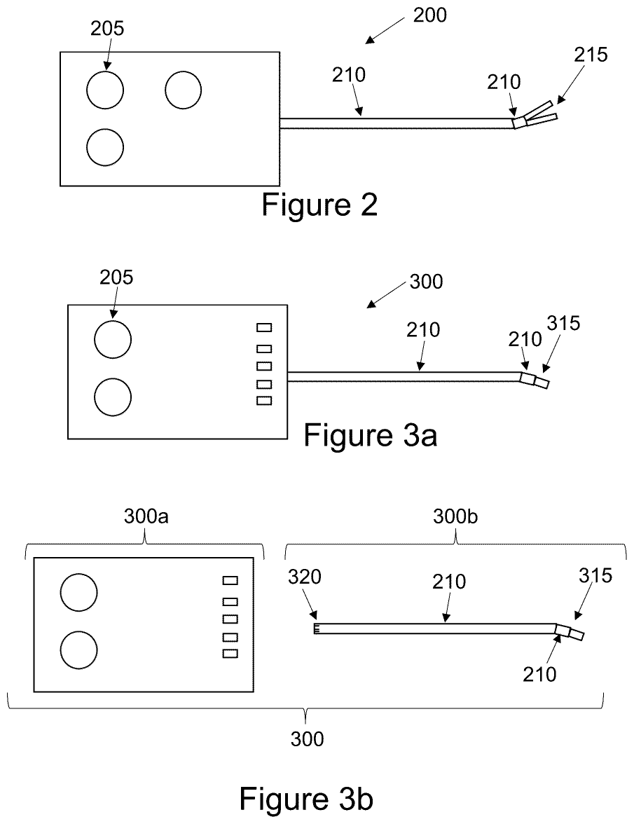

FIG. 2 illustrates a side view of an arthroscopic instrument 200 in the form of an arthroscopic grasper, according to an embodiment of the present invention. The instrument 200 may be similar or identical to one of the arthroscopic instruments 135 of FIG. 1.

The arthroscopic instrument 200 includes a plurality of drive elements 205, which are configured to engage with corresponding drive elements of the modular end plates of the arms 130, such that the system 100 may control the arthroscopic instrument 200 through rotation of the drive elements on the modular end plates.

The instrument 200 includes internal cabling, coupled to gears of the drive elements 205 and internal pulleys, to cause the instrument 200 to bend and actuate. Two of the drive elements 205/cables may be used to control bending of the instrument 200 (i.e. relative movement of components 210 around a joint), and a third drive element cable may be used to control a grasper 215 at a tip of the instrument 200. While not illustrated, the cable may cause the instrument 200 to bend using internal pulleys or the like.

The joint is located at a distal end of the instrument 200, to allow the instrument to change “pose” in the joint and work via a new approach. This enables the instrument 200 to work on areas that otherwise would not have been reachable.

The joint may include a pivot joint and a rotation joint, to enable the tip to bend in any direction with simply two cables.

FIG. 3a illustrates a side view of an arthroscopic instrument 300 in the form of an arthroscopic camera, according to an embodiment of the present invention. The instrument 300 may be similar or identical to one of the arthroscopic instruments 135 of FIG. 1.

The instrument 300 similarly includes internal cabling, coupled to gears of drive elements 205 and internal pulleys, to cause the instrument 300 to bend and actuate. However, instead of a punch, includes a camera 315 at the tip.

The camera 315 is coupled to a plurality of electrical contacts 320, such that in use, the camera 315 is electrically coupled to the system, to power the camera 315 and to send electrical signals back to the system in the form of live/streaming video imagery.

The instrument 300 comprises a reusable portion and a base portion.

FIG. 3b illustrates a side view of the arthroscopic instrument 300, in a separated configuration, according to an embodiment of the present invention. In particular, the instrument comprises a reusable base 300a, including an interface for providing images from the camera, and a disposal distal portion 300b, releasably coupled to and extending outwardly from the reusable base 300a.

The disposable distal portion 300b is configured to at least partly enter the body of a patient, and is this preferably provided in sterile form (e.g. in sterile packaging). The reusable base portion 300a does not enter the body of the patient, and is able to be covered by a drape, and thus need not be sterile.

The disposable distal portion 300b and the reusable base portion 300a are electrically and mechanically coupled when connected. A mechanical and electrical interface 325 is provided therebetween, and may include electrical contacts and mechanical drive members, such as cogs or gears, to cause the cables therein to move.

The reusable base portion 300a includes a camera control unit in addition to the interface, which may comprise a Bluetooth or similar interface.

Such configuration is less expensive than fully disposable surgical cameras, as the relatively expensive CCU and data interface may be reused, but alleviates the need to clean and sterilise parts, like fully reusable surgical cameras.

The modular end plates include powered drive elements and electrical contacts at the same predefined positions. The arthroscopic instruments 135 may then use any one or more of these powered drive elements and/or electrical contacts, based upon need. As an illustrative example, a fixed camera that is simply used for coarse patient location may not be coupled to any drive elements at all. Similarly, the modular end plates may include fluid inflow/outflow, suction and RF power connections.

While the above instruments are shown, the skilled addressee will readily appreciate that a wide range of instruments may be used due to the modular nature of the system 100. Generally, however, the arthroscopic instruments 135 will be less than about 5 mm in diameter, and more typically smaller (e.g. between 1.9 mm-4.0 mm in diameter). Similarly, the instruments may be between about 90 mm-170 mm length.

The instruments 135 may be curved, or able to bend, passively or through active bending, such as that outlined above.

Similarly, the instruments 135 may include internal functionality, such as processors, gearboxes or the like, to assist in their operation. As an illustrative example, arthroscopic shavers may be coupled to the modular end plate by an intermediate gearbox. Similarly, the instruments may include an identifier, e.g. an RFID identifier, to enable identification of the instrument.

The instruments 135 may be coupled to a fluid management system, to provide irrigation and regulate pressure, flow and to prevent heat dissipation into surrounding tissue, e.g. in the case of cauterisation or other heat generating instruments.

In addition to performing a function in surgery, in some embodiments the arthroscopic instruments 135 include sensors, such as temperature sensors, and provide data from the sensors to the system 100 through the modular end plates. This data may then be used to control systems, such as a fluid management system, as outlined below.

The instruments 135 will generally be sterile, either in the form single-use instruments or sterilised re-usable instruments. It is not practical to sterilise the arms 130. As such, single use and sterile drapes are placed over the arms.

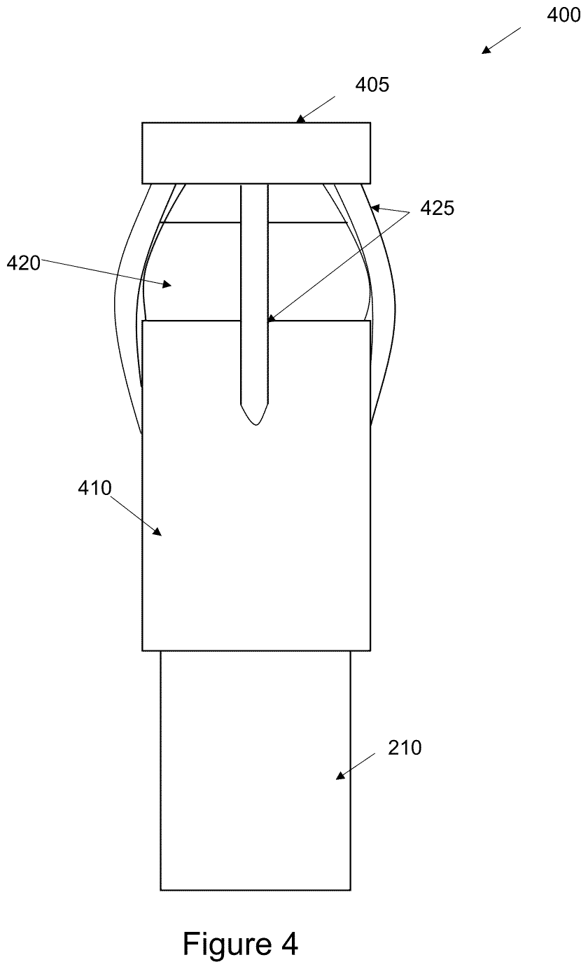

FIG. 4 illustrates a side view of an articulable joint 400 of an arthroscopic instrument of the system 100.

The articulable joint 400 is associated with a movement component 210 and forms an end of the surgical arms 130 to which arthroscopic instruments 135 are attached.

The articulable joint 400 includes a sheath 410 including a first end which fits around an end of the movement component 210, and a second end which receives a ball 420. The ball engages with an end cap 405 and enables rotary movement of the end cap 405 relative to the movement component 210. A variety of elements may then be attached to the end caps 405 to perform different functions.

The end cap 401 is secured in position over a portion of the ball 420 by supports 425. The supports 425 are positioned away from the ball 420 such that they do not impair rotary movement of the ball 420. The articulable joint 400 provides an alternative way of providing movement to instruments than the pulley/cable systems described above.

As outlined above, the system 100 may include sterile drapes over the arms 130.

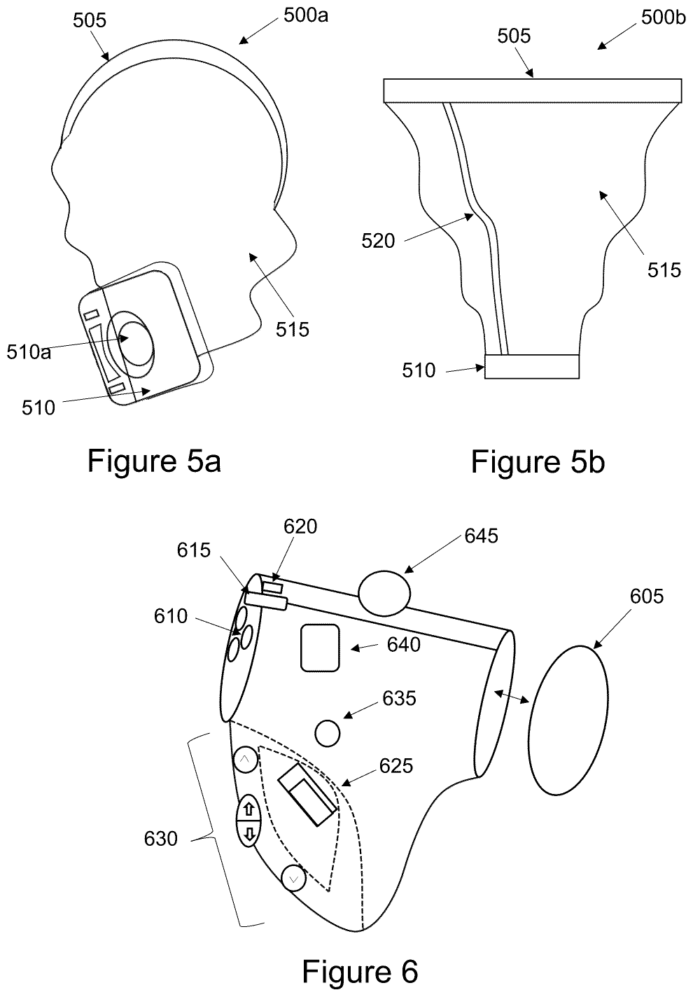

FIG. 5a illustrates a perspective view of a sterile drape 500a of the system 100, according to an embodiment of the present invention.

The drape 500a includes rigid upper and lower mounts 505, 510, and a flexible sheath 515 extending therebetween.

The rigid upper mount 505 is adapted to be received intermediate the frame 120 and the arm 130 (i.e. at the arm mount on the frame 120), and the rigid lower mount 510 is adapted to be received intermediate the arm 130 and the arthroscopic instrument 135 (i.e. at the modular end plate).

In particular, the rigid upper and lower mounts 505, 510 include apertures 510a therein through which the connectors of the arm 130 and instruments 135 extend. The flexible sheath 515 provides a continuous tube between the apertures 510a of the rigid upper and lower mounts 505, 510, and thereby encompasses the arm 130 its entirety, and thus provides a sterile outer surface thereof.

In some embodiments, the drapes may include tubing which is connectable to a fluid inflow/outflow system, suction system and/or an RF energy system, as described below.

FIG. 5b illustrates a simplified side view of a sterile drape 500b, according to an embodiment of the present invention. The drape 500b may be similar to the drape 500a but includes internal tubing 520.

The internal tubing 520 is for transporting fluid to or from a patient during surgery. The fluid may comprise saline or another intraoperative fluid. The skilled addressee will, however, readily appreciate that several tubes may be provided for various purposes, including suction tubes, as well as tubes not necessarily for fluid transport, such as tubes containing electrical wiring.

The internal tubing 520 extends between the rigid upper and lower mounts 505, 510, and interfaces for the tubing 520 are provided at the rigid upper and lower mounts 505, 510. In some embodiments, the rigid upper and lower mounts 505, 510 are provided such that they align with corresponding ports of the system 100 to simplify the installation of the tubes. As an illustrative example, the upper and lower mounts 505, 510 may include a locking mechanism, configured to lock the upper and lower mounts into place.

As outlined above, the surgical arms 130 include modular end plates, to which the arthroscopic instruments 135 are coupled.

FIG. 6 illustrates a simplified view of a modular end plate 600 of the system 100, according to an embodiment of the present invention.

The modular end plate 600 is coupled to the surgical arm 130 by an attachment portion 605, which includes a variety of mounting interfaces. The modular end plate 600 becomes a distal end of the surgical arm 130.

The modular end plate 600 includes a plurality of drive elements 610, configured to engage with and drive corresponding drive elements of the arthroscopic instrument 135. Three drive elements 610 are illustrated, but the skilled addressee will readily appreciate that any number of drive elements may be used.

Similarly, the modular end plate 600 includes an electrical connector 615, configured to engage with a corresponding electrical connector of the arthroscopic instrument 135.

A drape connection 620 is provided adjacent to the drive elements 610 and electrical connector 615, to enable a drape to be coupled to the end of the surgical arm, to thereby cover the surgical arm 130.

A plurality of interface buttons are then provided, to enable a person to interact with the modular end plate, as outlined below.

Initially, a manual override button 625 is provided, to enable the user to manually override movement of the surgical arm 130. A plurality of manual control buttons 630 are then provided, including an up button, and down button, a pitch up button, and a pitch down button. Such buttons are particularly useful for coarse movement of the arm.

A “save position” button 635 is provided, to enable the system to save a position of the arm 130. The position may then be reused later, or saved for the purpose of record keeping.

A “primary action” button 640 is provided to enable one or more primary action functions to be performed.

Finally, a 9-dimensional D-pad 645 is provided, to enable fine movement of the arm. The 9-dimensional D-pad 645 includes forward, backward, left and right, together with diagonal directions therebetween. A central portion of the D-pad 645 may be pressed to provide alternative functionality to the above functions. The functions may move the arm left, right, forward and backward, as well as yaw left, yaw right, roll left and roll right. Furthermore, up, down, pitch up and pitch down may be provided.

The skilled addressee will, however, readily appreciate that any suitable interface may be used to provide such functions.

Now turning back to FIG. 1, the system 100 includes a fluid management system 175, to regulate the flow of fluid (e.g. saline) directly to the joint via the instruments 135 and thus into the joint. The fluid management system 175 include a pump, pressure sensors and control valves (e.g. pinch valves) to regulate pressure and flow to the instruments. The fluid management system 175 may include one or more lines, and in the case of multiple lines, each line may be regulated independently.

A joint is generally sensitive to thermal changes and fluid pressure over the course of a procedure. When using ablation wands or rotary cutters/shavers, for example, there is almost always a change in temperature, flow and pressure within the joint. Temperatures above 50 degrees have been associated with cartilage cell death and high fluid pressure has been associated with swelling, compartment syndrome and risk of the limb. Similarly, alterations in flow can collapse the joint and lead to collisions between the joint surface and the instruments.

As such, the fluid management system may be configured to ensure that temperatures and pressures remain within certain thresholds.

The system 100 further includes a suction system 180, that is used for fluid outflow, and may be coupled to an external suction system, which manages the suction and handling of fluids.

As outlined above, the instruments 135 include sensors. In some embodiments, the fluid management system 175 utilises data from the sensors to regulate joint temperature, pressure and visibility by adjusting operating pressure and/or flow rate.

In preferred embodiments, the fluid management system 175 includes one or more peristaltic pumps. Sterile saline bags are coupled to the fluid management system 175, and the system is primed prior to use. The suction tract operates in a similar manner, but is either emptied into a suction reservoir or connected to a waste port, such as a wall suction port within the operating theatre.

The fluid management system 175 and the suction system 180 are provided within the articulable arm 125 to avoid or reduce the number of external hoses.

Finally, the system 100 includes a Radio Frequency (RF) energy generator 185 which coordinates and controls the creation of RF energy, for use by one or more of the instruments 135.

The RF energy generator 185 is a modular component of the system 100, and is thus installable when needed, and may be replaced with like or different systems.

The RF energy generator works to generate RF signals for the purpose of Radiofrequency Ablation (RFA) at an instrument 135. The RF signal is coupled to the instrument 135 through internal wiring in the arm 125.

The system 100 may include a variety of other modules, coupled thereto, according to need.

While not illustrated, the system 100 includes a central controller to control various aspects of the system. The central controller may comprise one or more processors, memory coupled to the processors, the memory including various instruction code to perform the functions of the system.

The central controller may thus control movement and operation of the instruments 135, receive data relating to the surgery (e.g. joint temperature and pressure), data of the system (e.g. pump pressure, flow rate), and provide a coordinated control of the various components.

As an illustrative example, in case of an RF wand being used, the system may control fluid irrigation rates and RF energy generation based upon temperature data relating to the joint to avoid tissue damage.

As outlined above, the system 100 includes a camera, for capturing image data internally in the joint. Due to the limited size of the instruments 135, the camera is a mono-optic camera.

One problem with traditional mono-optic camera systems is that it is difficult to perceive depth in such systems, which may result in misinterpretation of image data, which is clearly undesirable.

In some embodiments, the image data from the camera is used to create a depth map, and from this 3D video from the 2D source, as well as contour lines and/or depth data (or a derivative thereof) is overlaid over the image.

In one embodiment, multiple images of an area from different points are processed together to create depth maps. As the instruments 135 move slowly and in a well-defined manner, images of the joint may be captured from slightly different angles as the instrument moves therethrough. As the patient/joint is generally static, these images may be used together to create a depth map, even though the images were captured at slightly different points of time.

Several different algorithms may be used for such purpose, but will generally be based upon geometry and differences between images from slightly different angles. In short, common points are identified across images, and relative translation of these points between images is used to estimate depth.

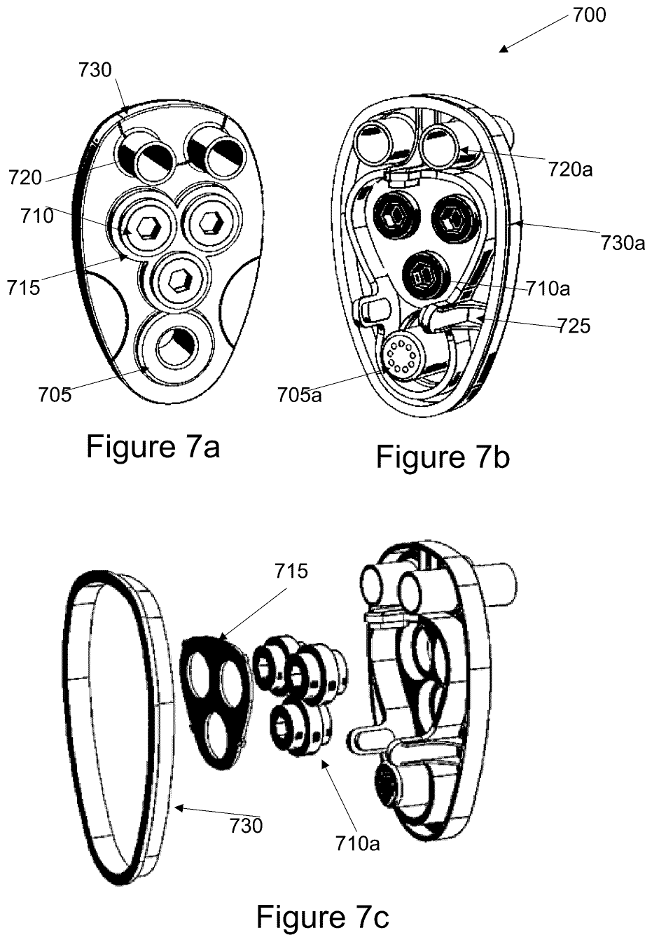

As outlined above, the arthroscopic instruments 135 are coupled to the surgical arms 130 by modular end plates. FIG. 7a illustrates an end plate 700 for the arthroscopic system 100. FIG. 7b illustrates an attachment portion of a modular end plate 700 for the arthroscopic instrument of the system 100. FIG. 7c illustrates an exploded view of the end plate 700 and attachment portions of FIGS. 7a and 7b.

The end plate 700 includes a receiving portion 705 for receiving an attachment portion 705a. Three motors 710a are received in corresponding motor couplings 710. Best illustrated by FIG. 7c, a retention plate 715 is positioned over the three motors 710a and corresponding motor couplings 710 to secure the motors 710 in place. Connection means 720 are configured to be received in connection means 720a to thereby connect the end plate and the attachment portion. As best illustrated by FIG. 7b, attachment portion includes struts 725 providing support.

As best illustrated by FIG. 7c, a retention clip 730 is secured over the attachment portion. The retention clip 730 is configured to retain a portion of flexible sheath 515 over the attachment portion so as to maintain sterility of the arthroscopic instruments 135 and operative zone.

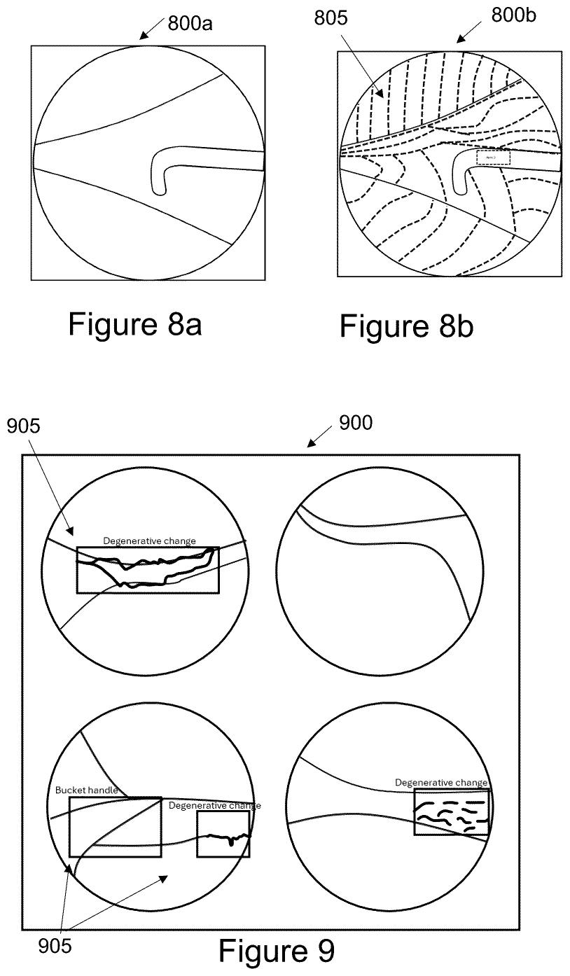

FIG. 8a illustrates a screenshot 800a of a camera image from the system 100, without any image overlay. As can be seen from the image, it can be very difficult to perceive depth, particularly in relation to relatively smooth surfaces.

FIG. 8b illustrates a screenshot 800b of a camera image from the system 100, with contour lines 805 overlaid onto the image.

The contour lines 805 are overlaid onto the image using sequences of dots, which may be colour coded. The use of dots is, however, only one example of how contour lines may be displayed, and the skilled addressee will readily appreciate the solid lines may instead be used.

In other embodiments, shading, colouring or the like may be used to represent depth and/or distance in an image. Alternatively again, the image may be labelled with depth cues in a wide range of manners.

In addition to creating depth maps, the images may be processed in a variety of ways prior to presentation to the user.

As an illustrative example, images may be flipped and/or scaled, to provide a more intuitive user experience. In such case, instead of operating based upon inverted views, the images may be flipped and/or scaled such that a consistent work environment may be provided to surgeons.

Similarly, image processing may be used to improve image quality, e.g. to improve contrast, focus, white balance, resolution. As movement of the camera is carefully controlled, the use of super-resolution techniques may be simplified and multiple images may be augmented to produce an image of higher resolution than the camera itself.

In some embodiments, image data may be classified during use. This classification may be performed manually, and/or automatically, using Artificial Intelligence (AI).

In one embodiment, training data from past cases is used to create identification classifiers (e.g. through Haar classifiers or similar). These identification classifiers may then be used to automatically classify anatomy of the joint.

In one embodiment, the system is configured to automatically identify “not normal anatomy”, and label same using image overlays or the like, using the classifiers.

FIG. 9 illustrates a screenshot 900 comprising four different image screens of the system 100 (i.e. images viewed by the surgeon during surgery), according to embodiments of the present invention.

The top left image includes an area marked with a bounding-box 905 and an associated label indicating “degenerative change”. As outlined above, the system may use identification classifiers to automatically identify such anatomy.

The top right image does not include any box or label, and thus illustrates the situation where no “not normal anatomy” has been identified.

The bottom left image includes multiple bounding-box elements 905, each with a different label.

Finally, the bottom right image includes a single bounding-box element 905, with an associated label.

The skilled addressee will readily appreciate that anything may be identified using such classifiers, include anatomy that is normal, as well as non-anatomy related aspects (e.g. equipment).

In addition to the manual control of the instruments, outlined above, the system 100 may include one or more automated functions, to assist the surgeon in performing operations.

As an illustrative example, the system 100 may perform automated tracking of a first instrument 135 with a second instrument 135. In such case, the first and second instruments may function in a “master and slave” type arrangement, where movement of the master causes the slave to follow.

Similarly, the system 100 may automatically move of one or more of the instruments 135 according to a desired target or path. The instrument 135 may move according to a desired location, and using object avoidance based upon image recognition. Alternatively, the instrument 135 may simply follow a path taken by another instrument 135.

Similarly, the system 100 may perform autonomous execution of one or more programs, such as milling. As an illustrative example, the surgeon may select one or more areas on which a task or function is to be performed. Parameters may also be defined for the task or function.

In one embodiment, an area may be selected by defining a bounding box using a user interface. A milling operation may then be selected for the area, upon which milling is automatically performed.

The task or function may include a repeated task (loop), or the operation of multiple sub-tasks. The operation of the repeated or sub-tasks may be performed without further interaction from the surgeon.

The task or functions may be performed by sending movement data to the arms in a particular sequence, or by setting parameters and initiating functions for the system.

The system 100 may be further configured to monitor tasks performed by a surgeon, and perform the task later. In such case, the system may monitor positional, visual, environment and operational data to train an Al program to perform such tasks autonomously.

Furthermore, the system 100 may monitor operative and instrument data to create inventory adjustments, make purchase orders and support logistics.

As outlined above, the frame enables accurate relative positioning of instruments 135, even with different portals are used. In some embodiments, the surgeon may use a graphical user interface to view the relative position of instruments with reference to each other.

The graphical user interface may illustrate a portion of the patient, and illustrate the positions of the instruments with reference to each other and relative to the patient. Such configuration enables the surgeon to get a quick overview of the location of various instruments during surgery.

In some embodiments, image data is captured of an external anatomy of the patient. This may be performed using a camera associated with the frame. This image data may then be used to identify a location of the patent relative to the system 100.

In some embodiments, markers or markings may be made on the patient, which are then used as reference points by the system. As an illustrative example, portal sites may be identified on the patient using markings, which are identified by the system.

The movement of the arms 130 may be controlled and coordinated in any suitable manner. In some embodiments, the control of the arms is performed with the use of forward kinematics and/or inverse kinematics.

As outlined above, the system 100 is modular, and enables the use of a variety of different arms and instruments, according to need. In some embodiments, the arms and/or instruments are registered to the system 100, e.g. using a serial number, a QR code, or RFID.

Upon registration, the system is configured to the arm or instrument, e.g. by obtaining parameters from a data store relating to the arm or instrument. Similarly, a command interface may be defined between the arm or instrument and the controller of the system.

While FIG. 1 illustrates three arms 130 and instruments 135, the skilled addressee will readily appreciate that any suitable number of arms and instruments may be used.

In addition to the controls and features described above, the system may be configured and re-configured in a number of different ways. For example, the system (e.g. using the console) may enable adjustment of the strength of the system, speed of the system, or even to manually reposition portions of the system (e.g. arms).

In addition to assisting a surgeon in performing surgery, the system 100 may be configured to capture operative data and document the surgery. Such operative data may include image data captured during surgery, as well as time-sequenced data of the system and instruments.

In some embodiments, the system may be configured to further capture data from the surgeon directly, such as a microphone configured to record speech of the surgeon, and or data entry forms, enabling the surgeon to document the surgery (e.g. clinical records and note taking).

The system may then share this data with one or more applications, or third-party systems. As an illustrative example, clinical records may be automatically uploaded to a clinical records system.

In some embodiments, QR code based pairing may be used to link a surgery to a specific surgeon. In such case, the surgeon may have an application installed on a smart device, which is configured to scan a QR code associated with the system. The QR code may be displayed on a screen and updated over time, to provide data security.

Alternatively again, accounts may be associated with the system 100 directly.

As will be readily appreciated by the skilled addressee, aspects of the system 100 may have varied shape and/or function without deviating from the scope of the present invention.

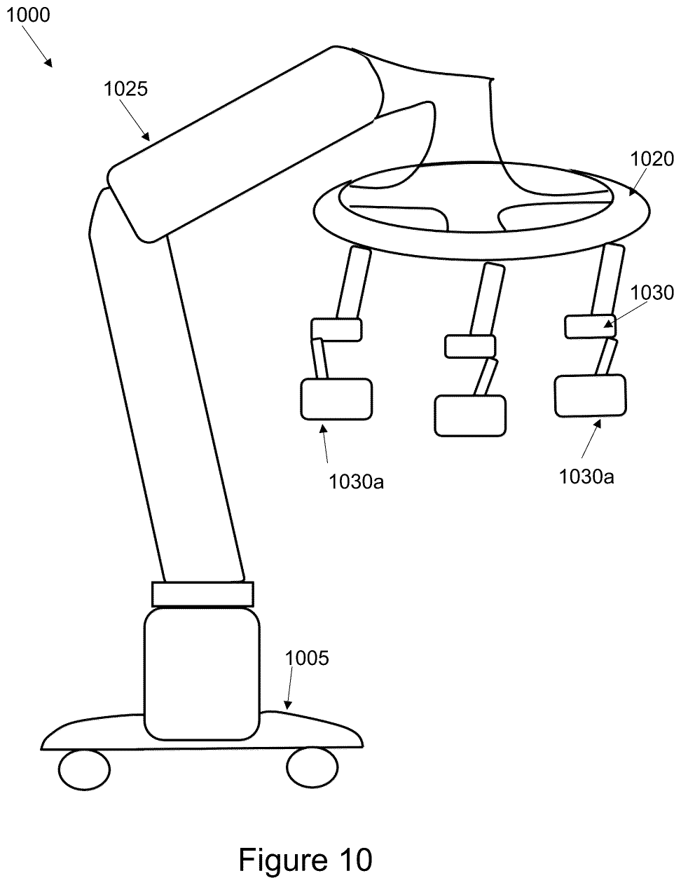

FIG. 10 illustrates a perspective view of a portion of an arthroscopic surgery system 1000, according to an alternative embodiment of the present invention.

The arthroscopic surgery system 1000 is similar to the system 100, and includes a wheeled base 1005, an articulable arm 1025 extending upwardly therefrom, and a frame 1020 from which a plurality of surgical arms 1030 extend.

No arthroscopic instruments 135 are illustrated in the system 1000, but instead modular mounting plates 1030a are provided on ends of the arms 1030 to enable different instruments to be installed, as needed.

The frame 1020 is annular in shape, and is rotatable.

As outlined above, a user interface is provided to enable the surgeon to remotely control the arthroscopic instruments. The user interface may be provided in a headset, or a display screen, as outlined above.

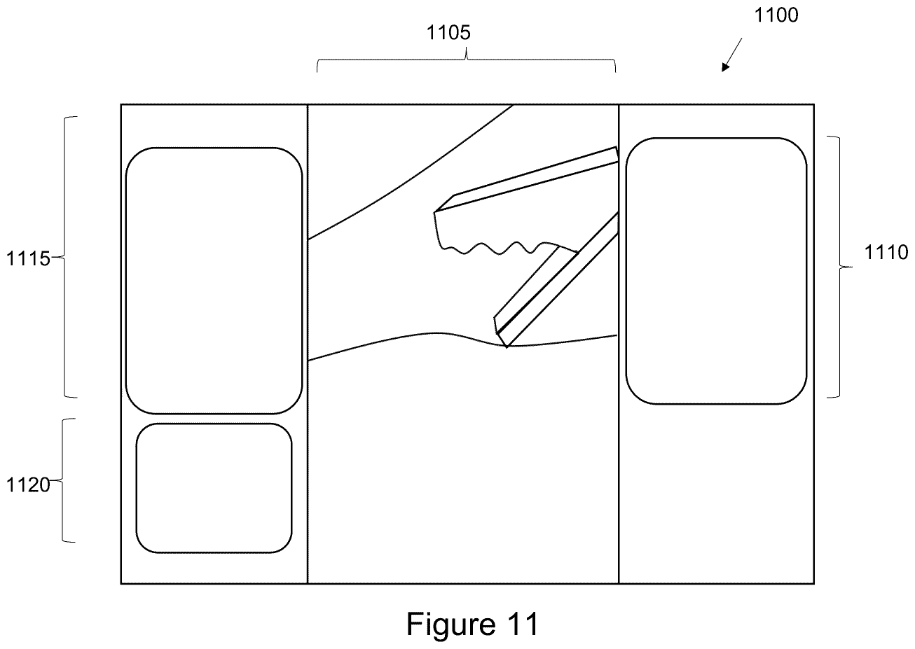

FIG. 11 illustrates a screenshot of a surgical screen of an arthroscopic surgery system, according to an embodiment of the present invention.

The surgical screen includes a live image element 1105, for showing an image from an arthroscopic camera. The image element 1105 is updated in or near real time, and includes a variety of configuration elements for adjusting the image, such as white balance, contrast, brightness, focus, adding depth cues/contour lines, or automatically generating and displaying a 3D image from the 2D image data and depth information.

The surgical screen further includes an instrument configuration element 1110, through which the instruments may be configured. This may include setting an instrument as a master instrument, and other instruments as slave instruments that follow the master instrument.

A fluid/electrical control element 1115 is provided, which displays temperature and electrical data and enables the setting of alarms based thereon. As an illustrative example, the element 1115 enables the setting of alarms when fluid temperature or pressure is above a certain threshold.

The surgical screen includes a map element 1120, showing relative positioning and configuration of the system according to a coordinate system.

The skilled addressee will readily appreciate that the coordinate system includes a variety of other functions, such as configuration functions, menus, settings, documentation elements, status elements and the like.

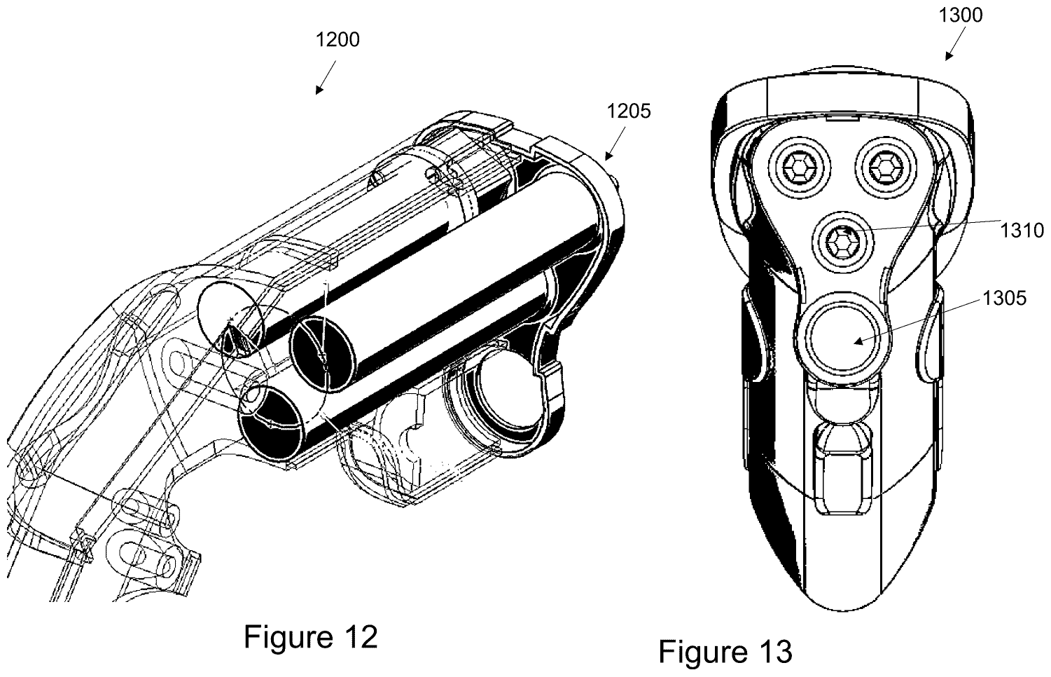

FIG. 12 illustrates a mounting end for an arthroscopic instrument including a hand grip of the system 100.

The mounting end 1200 includes a mounting plate 1205 configured to engage with a hand grip 1300 such as that illustrated in FIG. 13. The hand grip includes a rotary joy stick 1305 and motors 1310,

In use, it is envisioned that the hand grip 1300 together with the mounting end 1200 is affixed to a surgical arm 130. The hand grip 1300 is then gripped by a surgeon and moved to an appropriate position. The hand grip 1300 is linked to one or more of the surgical arms 130 such that movement at the hand grip 1300 causes the corresponding movement of the remaining surgical arms 130. The skilled addressee will appreciate that such configuration is useful for initially positioning the surgical arms 130 at an operative site.

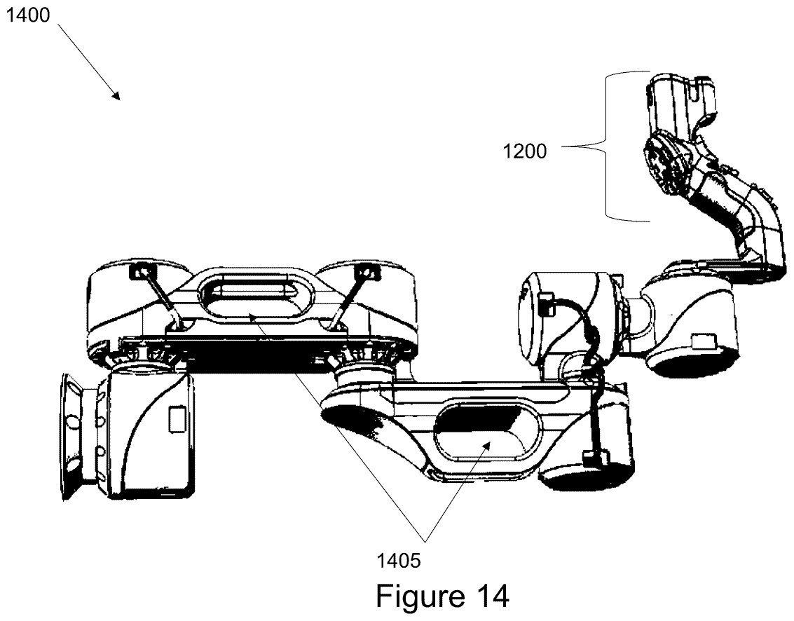

FIG. 14 illustrates a side view of a surgical arm 1400 of the arthroscopic system 100. As illustrated, the surgical arm 1400 includes a mounting plate configured to engage with the hand grip 1300. The skilled addressee will appreciate that the surgical arm 1400 may include any suitable arthroscopic instrument.

The surgical arm 1400 includes two hand grips 1405 for assisting in the movement of the surgical arm 1400. The hand grips are used to position the surgical arm 1400 in a suitable position. The suitable position may for example be at an operative site, or adjacent the articulable arm 125 of the system 100 when the system 100 is not in use.

The surgical arm 1400 includes the mounting end 1200 and mounting plate 1205. Such that the mounting plate 1205 may engage with the hand grip 1300.

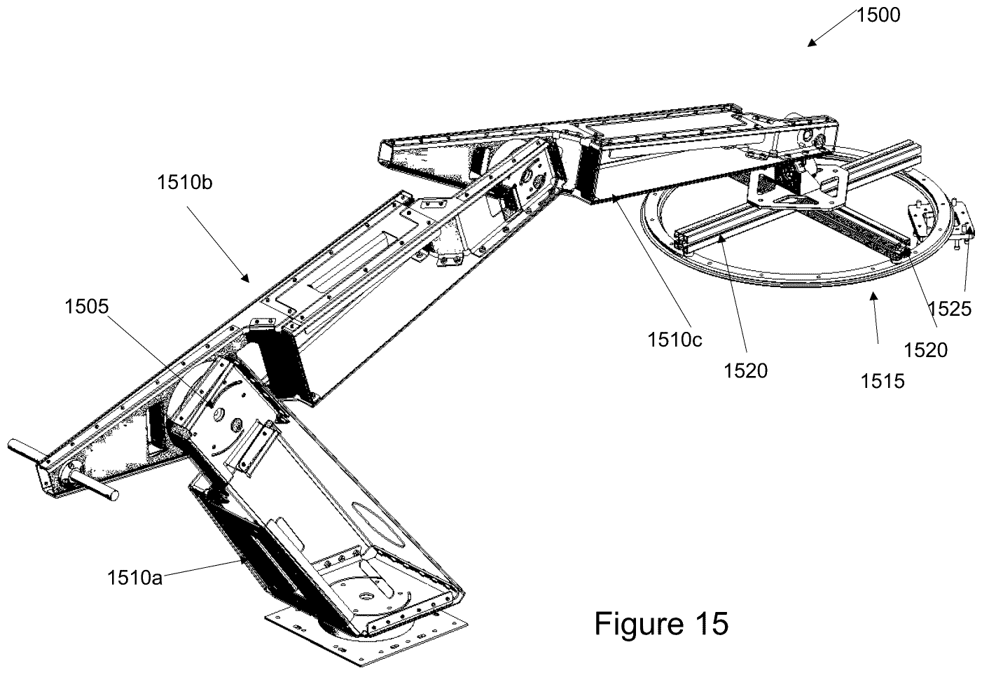

FIG. 15 illustrates side view of an articulable arm 1500 according to an alternate embodiment. The articulable arm 1500 is similar to the articulable arm 125 and frame 120 of system 100. The articulable arm 1500 includes pivot apertures and pivot pins enabling segments 1510a, 1510b, 1510c of the arm to pivot.

An annular frame 1515 includes two extrusions 1520 extending from a positioned at a center of the annular frame 1515, at right angles, and extending the diameter of the annular frame 1515. A movement assembly 1525 is mounted to a bottom position of the annular frame 1515 for connecting the surgical arms 130 to the annular frame 1515 such that they can move around the annular frame 1515.

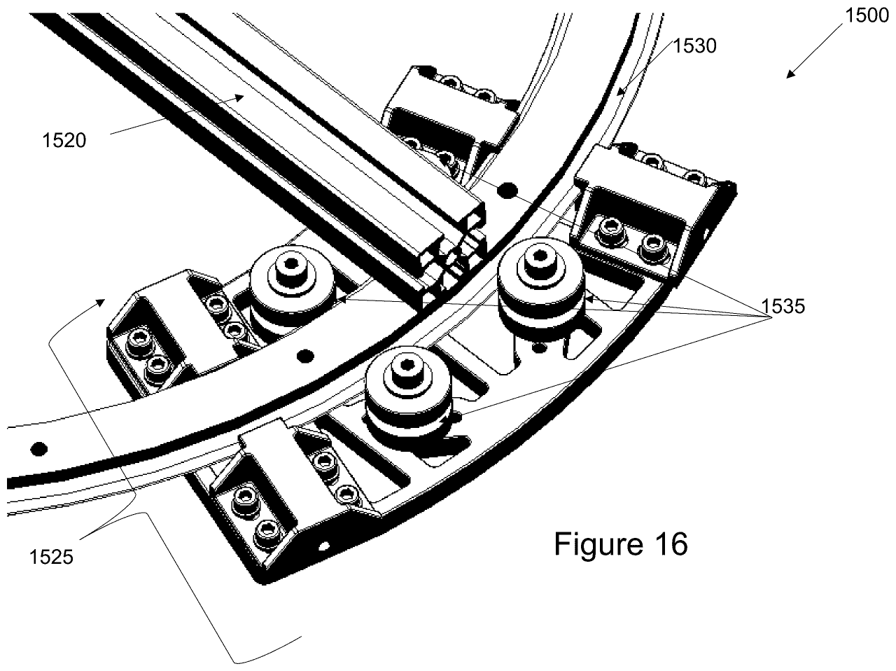

FIG. 16 illustrates an enlarged side view of the annular frame 1515 and movement assembly 1525 of FIG. 15. The movement assembly 1525 includes four rollers 1535 configured to engage with a track 1530 defined in the annular frame 1515.

Advantageously, arthroscopic surgery methods and systems are described above, which simplify arthroscopic surgery, and thereby reduce likelihood of damage or injury being caused from the surgery itself. Furthermore, as the methods and systems are simpler to use than traditional arthroscopic instruments, the barrier for entry into arthroscopy is reduced.

Unlike laparoscopic robots, which are big and bulky, and unable to be effectively used for arthroscopy, the arthroscopic surgery methods and systems described above, are not only suitably sized for arthroscopy, but also to enable instruments to access a joint from multiple portals, while remaining accurately positioned in a common reference point.

Furthermore, flow, pressure or temperature regulation is provided, rather than systems that promote energy dissipation into adjacent tissue, which would injure cartilage.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. An arthroscopic surgery system for assisting a surgeon in performing surgery comprising:
a frame configurable to extend above or beside at least a portion of a patient to be operated; and
one or more surgical arms extending from the frame, wherein each surgical arm is configured to work with a variety of different arthroscopic instruments;
wherein the one or more surgical arms are arranged to assist a surgeon when performing surgery and wherein the surgical arms and attached arthroscopic instruments are controllable by the surgeon whilst performing surgery on the patient.

2. An arthroscopic surgery system as claimed in claim 1, wherein each of the one or more surgical arms extend from a corresponding one or more plurality of locations on the frame.

3. An arthroscopic surgery system as claimed in claim 1, wherein the one or more surgical arms are removably coupled to the frame.

4. An arthroscopic surgery system as claimed in claim 2, wherein the one or more surgical arms are configured to be driven along a track provided on the frame.

5. An arthroscopic surgery system as claimed in claim 1, wherein the frame at least comprises an annular frame assembly for receiving one or more of the surgical arms.

6. An arthroscopic surgery system as claimed in claim 5, wherein at least the annular frame assembly is rotatable.

7. An arthroscopic surgery system as claimed in claim 5, wherein the annular frame assembly further comprises intersecting frame sections passing through a geometrically central region of the annular frame assembly, the frame sections being coupled to an articulable arm during use.

8. An arthroscopic surgery system as claimed in claim 7 wherein the intersecting frame sections are mutually perpendicular to each other.

9. An arthroscopic surgery system as claimed in claim 1, wherein at least one of the one or more surgical arms comprises a modular end plate which is configured to engage with a variety of different arthroscopic instruments.

10. An arthroscopic surgery system as claimed in claim 9, wherein the modular end plate includes one or more electronic, sensing and/or drive elements and/or interface elements configured to engage with corresponding elements of an arthroscopic instrument.

11. An arthroscopic surgery system as claimed in claim 9, wherein the modular end plate comprises one or more buttons which can be used to control the surgical arm.

12. An arthroscopic surgery system as claimed in claim 11, wherein the one or more buttons are configured to put the surgical arm into a manual mode wherein the surgical arm can be physically repositioned by a user.

13. An arthroscopic surgery system as claimed in claim 9, further comprising a hand unit associated with a surgical arm and/or a modular end plate, wherein the hand unit is positioned in a user's hand for the manual movement of one or more of the surgical arms.

14. An arthroscopic surgery system as claimed in claim 1, wherein at least one of the surgical arms has at least three degrees of freedom and more preferably at least six degrees of freedom selected from the group consisting of: (i) forward; (ii) back; (iii) up; (iv) down; (v) left; (vi) right; (vii) yaw; (viii) pitch; and (ix) roll.

15. An arthroscopic surgery system as claimed in claim 1, further comprising at least one drape configured to extend over at least one of the one or more surgical arms.

16. An arthroscopic surgery system as claimed in claim 1, wherein the one or more arthroscopic instruments include an identifier to allow the system to identify the arthroscopic instrument attached, in use, to a surgical arm.

17. An arthroscopic surgery system as claimed in claim 16, wherein the identifier comprises an electronic tag.

18. An arthroscopic surgery system of claim 1, wherein at least one of the arthroscopic instruments comprises a surgical camera.

19. An arthroscopic surgery system as claimed in claim 18, wherein the surgical camera comprises a reusable base portion including an interface for providing images from the camera and a disposable distal portion releasably coupled to the reusable base portion.

20. An arthroscopic surgery system as claimed in claim 1, further comprising a fluid management system including one or more pumps and valves to regulate fluid pressure and/or flow and/or visual acuity at a surgery site, wherein the one or more pumps and valves are connected to the surgery site by tubing.

21. An arthroscopic surgery system as claimed in claim 1, further comprising one or more consumables wherein the one or more consumables comprise a readable identifier and wherein the system further comprises a reader for reading the readable identifier and determining whether the one or more consumables are genuine consumables.

22. An arthroscopic surgery apparatus for assisting a surgeon in performing surgery comprising:
a frame configurable to extend above or beside at least a portion of a patient to be operated; and
one or more surgical arms extending from the frame, wherein each surgical arm is configured to work with a variety of different arthroscopic instruments;
wherein the one or more surgical arms are arranged to assist a surgeon when performing surgery and wherein the surgical arms are controllable by the surgeon whilst performing surgery on the patient.

23. A method of arthroscopic surgery, the method comprising the steps of:
providing an arthroscopic surgery apparatus comprising a frame and one or more surgical arms extending from the frame;
attaching an arthroscopic instrument to one or more of the surgical arms;
positioning the frame and/or attached arthroscopic instrument to extend above or beside at least a portion of a patient to be operated; and
utilising the one or more surgical arms to assist a surgeon when performing surgery;
wherein the method further comprises the surgeon controlling movement of the surgical arms and/or attached arthroscopic instruments whilst performing surgery on the patient by accessing a controller located at or adjacent the surgical arms.

24. An arthroscopic surgery system for assisting a surgeon in performing surgery comprising:
a frame configurable to extend above or beside at least a portion of a patient to be operated;
one or more surgical arms extending from the frame, wherein each surgical arm comprises a modular end plate configured to work with a variety of different arthroscopic instruments;
one or more arthroscopic instruments coupled, in use, to the one or more surgical arms via a modular end plate;
wherein each modular end plate is configured to provide mechanical control and/or electrical control of an arthroscopic instrument for surgery

25. A method of performing arthroscopic surgery comprising:
providing an arthroscopic surgery system for assisting a surgeon in performing surgery, wherein the arthroscopic surgery system comprises a frame configurable to extend above or beside at least a portion of a patient to be operated and one or more surgical arms extending from the frame, wherein each surgical arm comprises a modular end plate configured to work with a variety of different arthroscopic instruments;
attaching an arthroscopic instrument to each surgical arm via a modular end plate, wherein the arthroscopic instrument comprises an identifier;
identifying the arthroscopic instrument utilising the identifier; and
mechanically controlling and/or electrically controlling an arthroscopic instrument via the modular end plate.
